(19) United States
(12) Patent Application Publication
(10) Pub. No.: US 2009/0210005 A1
(43) Pub. Date: Aug. 20, 2009

(54) METHOD AND SYSTEM FOR DISPLACING HYOID BONE

(76) Inventors: Fred B. Dinger, III, San Antonio, TX (US); Jeffrey S. Wrana, San Antonio, TX (US); DONALD A GONZALES, San Antonio, TX (US)

Correspondence Address: FULBRIGHT & JAWORSKI L.L.P., 600 CONGRESS AVE., SUITE 2400, AUSTIN, TX, 78701, US

(21) Appl. No.: 12/372,218

(22) Filed: Feb. 17, 2009

Related U.S. Application Data
(60) Provisional application No. 61/029,808, filed on Feb. 19, 2008.

(51) Int. Cl. A61B 17/04

(52) U.S. Cl. 606/228; 606/232

(57) ABSTRACT

A system and method for moving a hyoid bone closer to thyroid cartilage in a patient. Systems and methods may comprise sutures, suture anchors, clips and grommets to move the hyoid bone closer to the thyroid cartilage.

### CROSS-REFERENCE TO RELATED APPLICATIONS

This application claims priority to U.S. Provisional Patent Application No. 61/029,808 filed on Feb. 19, 2008, the disclosure of which is herein incorporated by reference.

### BACKGROUND OF THE INVENTION

Obstructive sleep apnea is caused by the collapse of soft tissue in the airway during sleep resulting in cessation of breathing. Tissue collapse can either be retropalatal, retrolingual or both. Many methods are now available to treat the retropalatal obstruction, but treatment for retrolingual obstruction remains a difficult clinical challenge. One procedure that has shown promise is the hyoid suspension. The hyoid bone is a C-shaped bone in the upper/anterior neck that is attached posteriorly to the base of tongue. By moving the bone in a more anterior direction, the base of tongue will also move anteriorly and reduce the possibility of obstruction during sleep. Typically, the suture or wire is placed around the hyoid bone and pulled into proximity to the superior edge of the thyroid cartilage. Although the procedure has shown promise, especially when combined with a retropalatal procedure, few surgeons perform the procedure because of technical difficulty and potential complications. Complications occur because of the proximity of the laryngeal space to the posterior aspect of the hyoid bone. Because of this proximity, the suture or wire may erode through the mucosa and become exposed. Also, because of the circumferential tension placed on the hyoid bone, it often fractures resulting in a failed procedure. There are also several technical difficulties in performing a hyoid suspension. These difficulties include: tying a suture under tension, the ability to tailor the position of the hyoid bone relative to the thyroid cartilage, passing a suture through an ossified thyroid cartilage and stabilizing the “floating” hyoid bone.

Examples of existing systems and methods to used to secure a hyoid bone to thyroid cartilage are included in Appendix A, incorporated herein by reference.

### SUMMARY

Embodiments of the current invention provide a method of securing the hyoid bone to the thyroid cartilage using a suture or a modified suture and a combination of anchoring devices and/or grommets.

The anchoring devices can be suture anchors, which are well described in the prior art. For the purposes of this disclosure, a suture anchor is a metallic, biologic or polymeric device that consists of an anchor body, a bone fixation method and an attachment mechanism for the suture. Suture anchors can be fixed to the bone via a twisting motion (screw type) or via an axial driving motion (push-in type). Screw-in anchors can have various thread patterns or ridges for boney purchase. The push-in type anchors can be fixed via deforming barbs, deforming wings, toggling or lever actuated mechanisms. The suture can be attached so that it can slide with respect the suture anchor or it can not. Various rings, pin or eyelets are typically employed to secure the suture to the anchor body.

The approximation of the hyoid bone to the thyroid cartilage is achieved by the drawing together of the suture ends. This tightening decreases the overall length of the suture between the hyoid bone and the thyroid cartilage. Permanent displacement of the hyoid bone occurs with the suture loop length is secured. This can be accomplished via a standard suture knot or by other technologies the secure the suture without the use of a knot. These technologies are well described in the prior art as “knotless fixation systems” or “knotless suture anchors”. Non-limiting examples of such systems are disclosed in U.S. Pat. Nos. 5,658,313; 5,709,708; 6,143,017; and 6,156,039, each of which are herein incorporated by reference.

To prevent the suture from tearing through the thyroid cartilage when the hyoid bone is approximated, a device can be used to increase the bearing strength of the hole. An example of such a device is a grommet. This device would be placed at select locations in the thyroid cartilage and the suture would be placed through it.

Any number and combination of anchoring devices could be used depending access, anatomy and/or surgeon preference. FIGS. 1-12 depict various constructs that could be used.

### BRIEF DESCRIPTION OF THE DRAWINGS

FIG. 1 illustrates a perspective view of a hyoid bone and thyroid cartilage, according to exemplary embodiments of the present disclosure.

FIG. 2 illustrates a perspective view of a hyoid bone and thyroid cartilage with additional components secured, according to exemplary embodiments of the present disclosure.

FIG. 2A illustrates a perspective view of a suture anchor, according to exemplary embodiments of the present disclosure.

FIG. 2B illustrates a perspective view of a suture anchor, according to exemplary embodiments of the present disclosure.

FIG. 3 illustrates a perspective view of a hyoid bone and thyroid cartilage with additional components secured, according to exemplary embodiments of the present disclosure.

FIG. 4 illustrates a front view of a hyoid bone and thyroid cartilage with additional components secured, according to exemplary embodiments of the present disclosure.

FIG. 5 illustrates a perspective view of a hyoid bone and thyroid cartilage with additional components secured, according to exemplary embodiments of the present disclosure.

FIG. 6 illustrates a perspective view of a hyoid bone and thyroid cartilage with additional components secured, according to exemplary embodiments of the present disclosure.

FIG. 7 illustrates a perspective view of a hyoid bone and thyroid cartilage with additional components secured, according to exemplary embodiments of the present disclosure.

FIG. 8 illustrates a front view of a hyoid bone and thyroid cartilage with additional components secured, according to exemplary embodiments of the present disclosure.

FIG. 9 illustrates a perspective view of a hyoid bone and thyroid cartilage with additional components secured, according to exemplary embodiments of the present disclosure.

FIG. 10 illustrates a side and perspective view of a hyoid bone clip, according to exemplary embodiments of the present disclosure.

FIG. 11 illustrates a perspective view of a hyoid bone and thyroid cartilage with additional components secured, according to exemplary embodiments of the present disclosure.

FIG. 12 illustrates a front view of a hyoid bone and thyroid cartilage with additional components secured, according to exemplary embodiments of the present disclosure.

### DETAILED DESCRIPTION

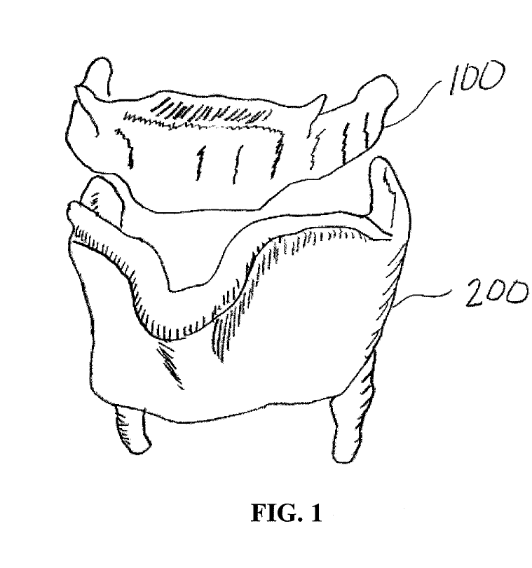

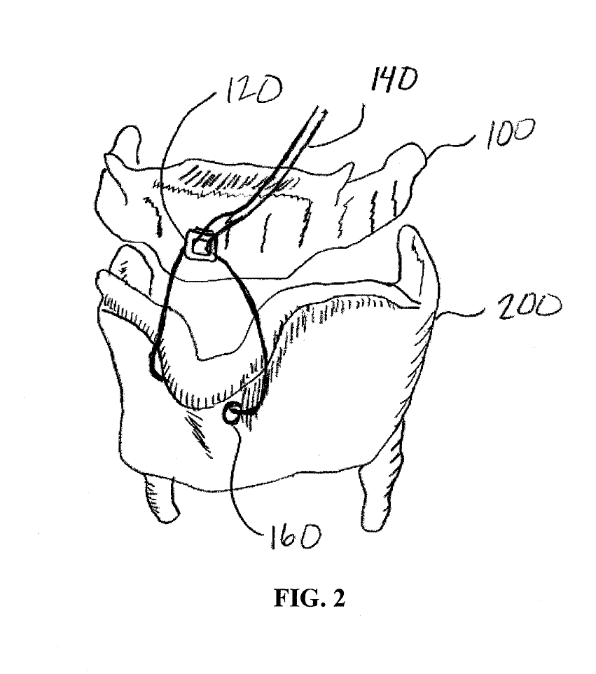

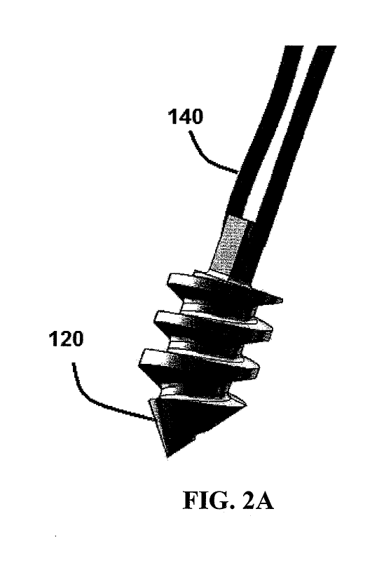

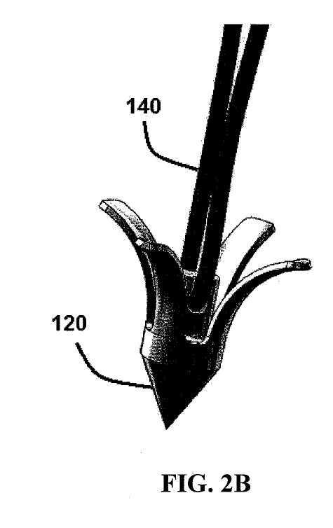

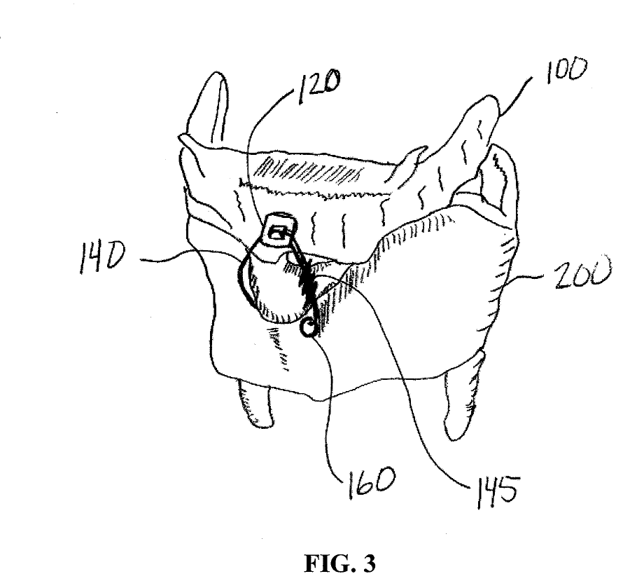

Referring now to FIG. 1, a hyoid bone 100 and thyroid cartilage 200 are initially spaced apart from each other, as is typical in a normal anatomy. In the exemplary embodiment shown, suture anchor 120 is installed in or secured to hyoid bone 100 and a suture 140 is passed through the suture anchor 120 and thyroid cartilage 140, as shown in FIG. 2. Exemplary embodiments of suture anchor 120 are shown in FIGS. 2A and 2B. The suture can then be shortened so that hyoid bone 100 is displaced towards thyroid cartilage 200, as shown in FIG. 3. In certain exemplary embodiments, suture 140 can be shortened by tying a knot 145 in suture 140. In other exemplary embodiments, suture anchor 120 is a knotless or self-locking suture anchor, so that a knot is not needed to secure suture 140 to suture anchor 120 and bring hyoid bone 100 closer to thyroid cartilage 200. In exemplary embodiments, one or more reinforcing members or grommets 160 may be placed in the thyroid cartilage where suture 140 penetrates the thyroid cartilage. In other embodiments, grommets 160 may not be used.

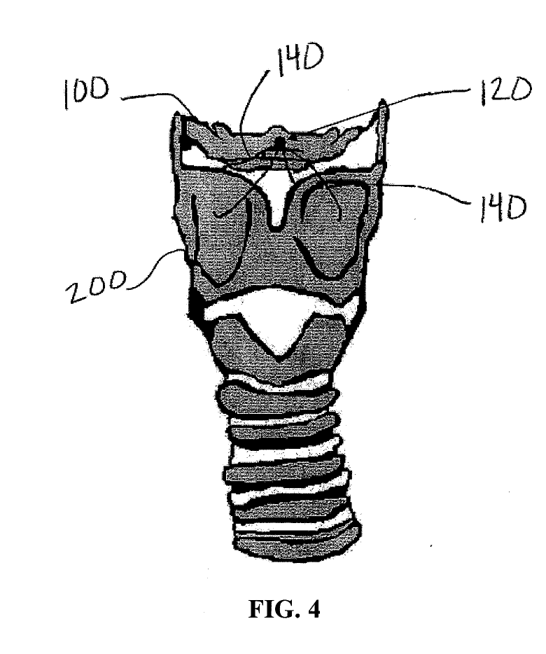

Referring now to FIG. 4, a system similar to FIG. 3 has been utilized to move hyoid bone 100 closer to thyroid cartilage 200. However in this exemplary embodiment, suture 140 passes through thyroid cartilage 200 in different locations than in FIG. 3.

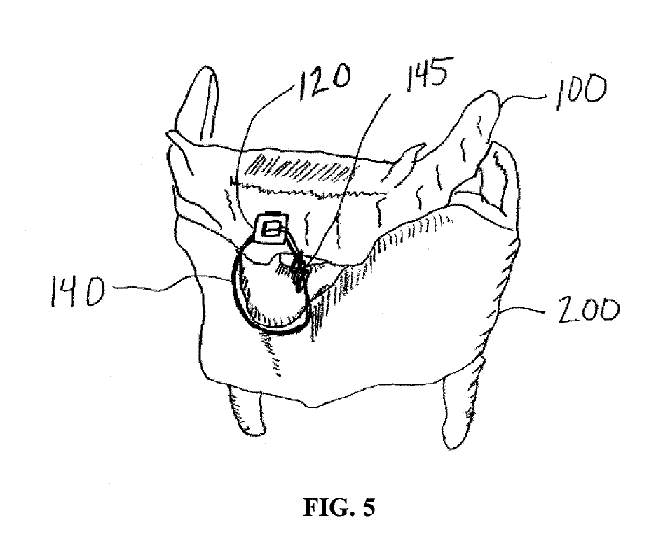

Referring now to FIG. 5, hyoid bone 100 has been displaced closer to thyroid cartilage 200 in a manner similar to that described in FIGS. 1-3. However, rather than passing suture 140 through thyroid cartilage 200, suture 140 is passed around a portion of thyroid cartilage 200. In the embodiment shown in FIG. 4, suture 140 is passed around a laryngeal prominence 210.

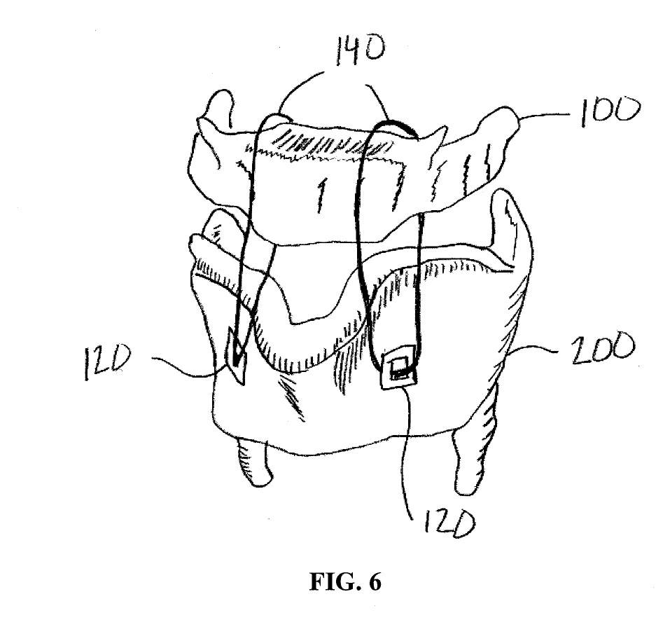

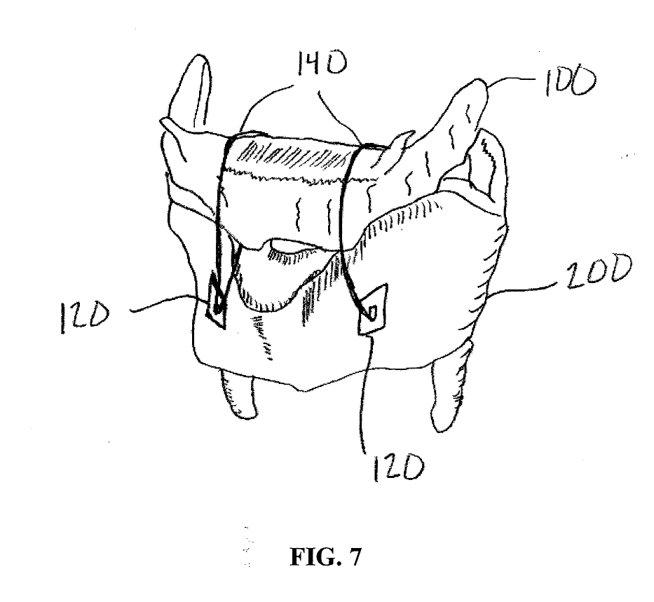

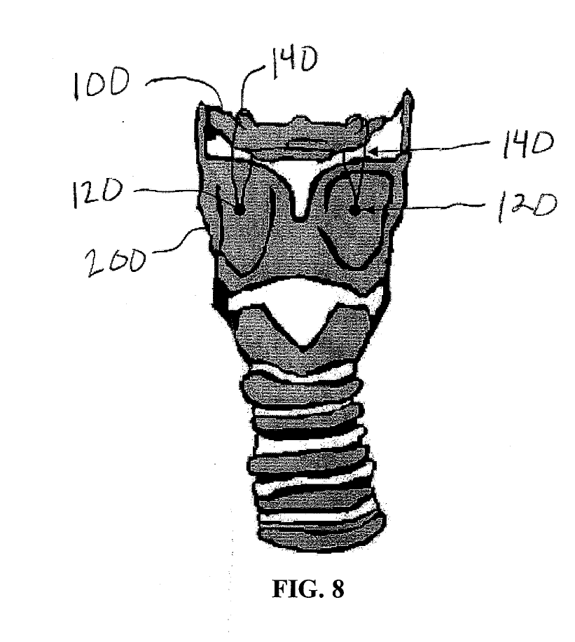

Referring now to FIGS. 6-8, hyoid bone 100 can be displaced closer to thyroid cartilage 200 by installing one or more suture anchors 120 into thyroid cartilage 200 rather than hyoid bone 100. In the exemplary embodiment shown, sutures 140 can be passed through suture anchors 120 and around hyoid bone 100. As shown in FIGS. 7 and 8, similar to previously-described embodiments, suture 140 may then be shortened to bring hyoid bone 100 closer to thyroid cartilage 200.

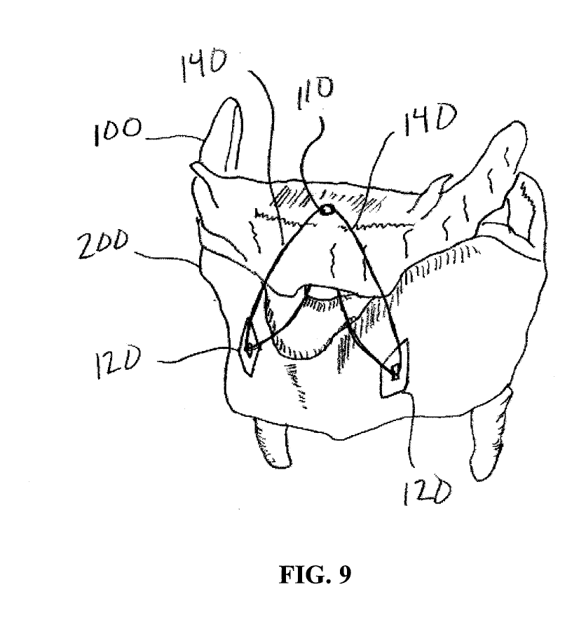

Referring now to FIG. 9, hyoid bone 100 has been brought closer to thyroid cartilage 200 in a manner similar to that described in FIG. 6. However, instead of passing suture 140 around hyoid bone 100, suture 140 has been passed through hyoid bone 100. A hole 110 may be drilled through hyoid bone 100 to facilitate passing suture 140 through hyoid bone 100. In certain embodiments, a grommet 160 may be used to reinforce hole 110. In other exemplary embodiments, a grommet 160 may not be used.

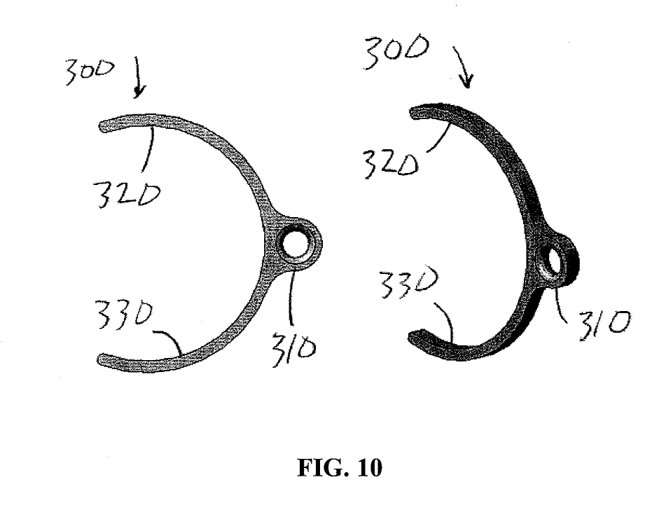

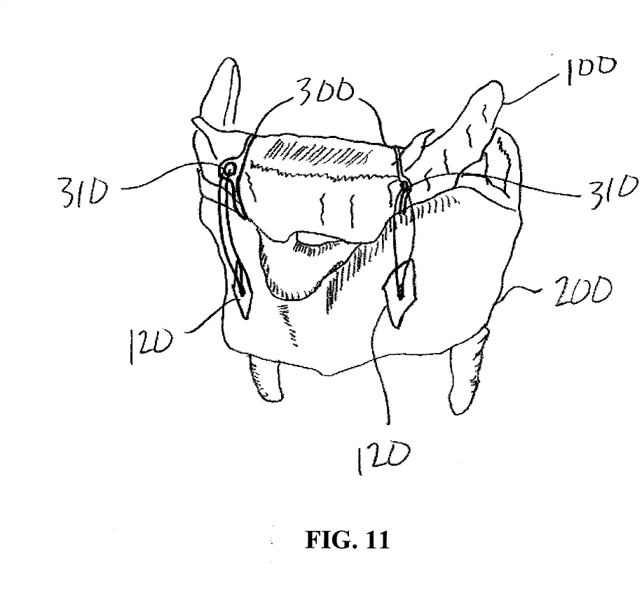

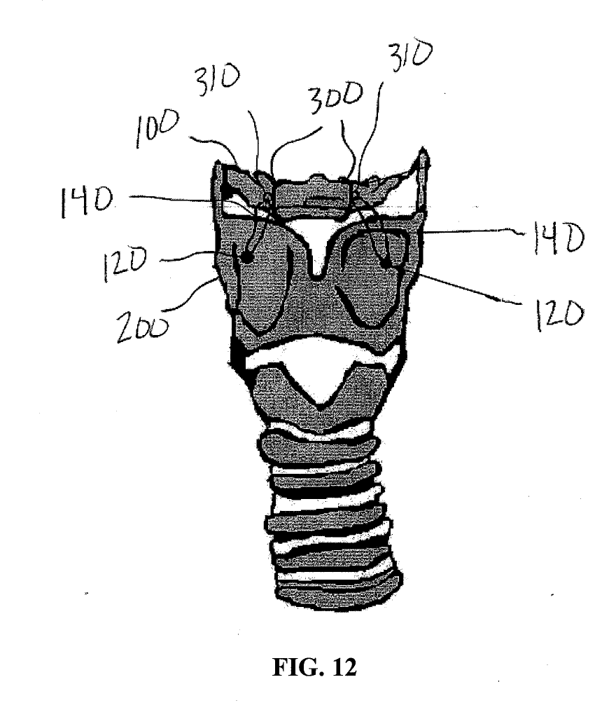

Referring now to FIGS. 10-12, a clip 300 may be secured to hyoid bone 100 in exemplary embodiments. Clip 300 may comprise an aperture or eyelet 310 and a pair extensions 320 and 330 that may be secured to hyoid bone 100. In exemplary embodiments, clip 300 may comprise a material such as nitinol or other material which can be relaxed from a pre-stressed state to secure clip 300 to hyoid bone 100. In other embodiments, clip 300 may be plastically deformed so that extensions 320 and 330 secure clip 300 to hyoid bone 100. In the exemplary embodiment shown, extensions 320 and 330 are curved, while in other embodiments, the extensions may be more linear. As shown in FIGS. 11 and 12, sutures 140 may be passed through eyelets 310 and suture anchors 120 to draw hyoid bone 100 closer to thyroid cartilage 200.

### Equivalents and Scope

The foregoing has been a description of certain non-limiting preferred embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims. For example, the different locations or members shown in various embodiments used to provide a coupling means for the hyoid bone and the thyroid cartilage may be combined in other exemplary embodiments. In addition, grommets may be used in exemplary embodiments of this disclosure that do not include grommets.

In the claims articles such as “a”, “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that embodiments of the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, embodiments of the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

What is claimed is:

1. A method of displacing a hyoid bone towards a thyroid cartilage, the method comprising:
securing at least one suture anchor to the thyroid cartilage;
passing a suture through or about the hyoid bone; and
coupling the suture to the at least one suture anchor.

2. The method of claim 1, wherein the at least one suture anchor is a self-locking suture anchor.

3. The method of claim 1, further comprising reducing the length of the suture between the hyoid bone and the suture anchor.

4. A method of displacing a hyoid bone towards a thyroid cartilage, the method comprising:
securing at least one suture anchor to the hyoid bone;
passing a suture through or about the thyroid cartilage; and
coupling the suture to the at least one suture anchor.

5. The method of claim 3, further comprising reducing the length of the suture between the thyroid cartilage and the suture anchor.

6. The method of claim 4, wherein the at least one suture anchor is a self-locking suture anchor.

7. The method of claim 4, further comprising:
installing a grommet in the thyroid cartilage; and
passing the suture through the grommet.

8. A method of displacing a hyoid bone towards a thyroid cartilage, the method comprising:
securing at least one suture anchor to the hyoid bone;
securing at least one suture anchor to the thyroid cartilage; and
coupling the at least one suture anchor in the hyoid bone to the at least one suture in the thyroid cartilage.

9. The method of claim 4, wherein the at least one suture anchor secured to the hyoid bone is a self-locking suture anchor.

10. The method of claim 4, wherein the at least one suture anchor secured to the thyroid cartilage is a self-locking suture anchor.

11. The method of claim 8 wherein a suture couples the at least one suture anchor in the hyoid bone to the at least one suture in the thyroid cartilage.

12. A method of displacing a hyoid bone towards a thyroid cartilage, the method comprising:
securing at least one clip to a hyoid bone, wherein the at least one clip comprises an eyelet;
securing at least one suture anchor to the thyroid cartilage;
passing a suture through the eyelet of the at least one clip and through the at least one suture anchor; and
coupling the suture to the at least one suture anchor.

13. A clip comprising:
a first extension;
a second extension; and
an eyelet, wherein the first extension and the second extension are configured to secure the clip to a hyoid bone during use.

14. A kit for displacing a hyoid bone, the kit comprising:
at least one suture anchor;
a suture;
a grommet; and
a clip configured for securement to a hyoid bone.
